(19) United States
(12) Patent Application Publication
(10) Pub. No.: US 2025/0043008 A1
(43) Pub. Date: Feb. 6, 2025

(54) FUSION PROTEIN OF INTERFERON (IFN) AND ANTI-PD-L1 ANTIBODY AND USE THEREOF

(71) Applicant: INSTITUTE OF BIOPHYSICS CHINESE ACADEMY OF SCIENCES, Beijing (CN)

(72) Inventors: Yangxin FU, Beijing (CN); Yong LIANG, Beijing (CN); Hua PENG, Beijing (CN)

(73) Assignee: INSTITUTE OF BIOPHYSICS CHINESE ACADEMY OF SCIENCES, Beijing (CN)

(21) Appl. No.: 18/820,339

(22) Filed: Aug. 30, 2024

Related U.S. Application Data
(63) Continuation of application No. 17/048,106, filed on Oct. 16, 2020, now Pat. No. 12,077,589.

(30) Foreign Application Priority Data
Apr. 16, 2018 (CN) 201810336178.1

(51) Int. Cl. C07K 16/28; A61K 38/21; A61K 39/395; A61K 47/68; A61P 35/00; C07K 14/555; C07K 14/56; C07K 14/565; C07K 14/57

(52) U.S. Cl. CPC C07K 16/2827; A61K 38/21; A61K 38/212; A61K 38/215; A61K 38/217; A61K 39/3955; A61K 47/6849; A61P 35/00; C07K 14/555; C07K 14/56; C07K 14/565; C07K 14/57; C07K 16/2818; C07K 2317/21; C07K 2317/24; C07K 2317/54; C07K 2317/55; C07K 2317/569; C07K 2317/622; C07K 2319/30

(57) ABSTRACT

An interferon (IFN)-anti-PD-L1 fusion protein, a pharmaceutical composition and a kit containing the same for treating tumors are disclosed. The fusion protein of the present invention can simultaneously target PD-L1 and IFN receptors, and the activation of IFN signals in a tumor microenvironment (TME) can enhance the PD-1/PD-L1 therapy against tumors by inducing stronger T cell activation. The anti-PD-L1 antibody can be used to specifically deliver immunomodulatory molecules to tumor tissues, and the fusion protein results in the generation of multiple feedforward responses, which can increase the targeting effect, reduce the toxicity, and enhance the response to IFN therapy, thereby maximizing the anti-tumor effect.

### CROSS REFERENCE TO THE RELATED APPLICATIONS

This application is a continuation application of U.S. application Ser. No. 17/048,106, filed on filed on 2020 Oct. 16. U.S. application Ser. No. 17/048, 106 is the national phase entry of International Application No. PCT/CN2019/082360, filed on Apr. 12, 2019, which is based upon and claims priority to Chinese Patent Application No. 201810336178.1, filed on Apr. 16, 2018, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBCHTN001-CONT-PKG_SequenceListing_20241025.xml, created on Oct. 25, 2024, and is 73,540 bytes in size.

### TECHNICAL FIELD

The present invention belongs to the technical field of genetic engineering and biomedical medicine, and in particular, relates to a fusion protein of an interferon (IFN) and an anti-PD-L1 antibody, a pharmaceutical composition and a kit containing the same, and use thereof in the treatment of tumor diseases.

### BACKGROUND

Programmed cell death protein 1 (PD-1) is a key immune checkpoint molecule. It can inhibit the TCR signal activation of T cells, which reduces the strength and duration of the immune response. A ligand of PD-1, PD-L1, is generally up-regulated in tumor cells, which is one of the immune evasion mechanisms of tumors. PD-1/PD-L1 blocking therapy (PD therapy) can induce a variety of cancer patients to produce a lasting immune response to tumor cells. In practice, however, objective and effective immune responses are only observed in a small number of patients undergoing PD therapy. In addition, drug resistance occurring in acquired PD therapy is also receiving wider attention, while its mechanism is still unclear. Therefore, it has become the top priority of PD therapy-related research to study and analyze why certain tumors cannot respond to or become resistant to PD therapy.

Some scholars have proposed that PD therapy can effectively control tumors because T cells release immunosuppressive signals. It is reported that the excellent efficacy of PD therapy is related to a sufficient number of tumor infiltrating lymphocytes (TILs). Even in the case of a large number of TILs, however, PD therapy alone may not be effective in (re)activating tumor-specific T cells. In these cases, it may need to block other negative synergistic inhibitors or up-regulate stimulus signals to induce the (re)activation of T cells. There are still many unanswered questions about the signaling molecules that can effectively promote T cell immunity.

Type I IFNs include IFNα and IFNβ, which activate T cells by promoting the maturation of dendritic cells (DCs) that process and present antigens, thereby acting as a bridge between natural immunity and adaptive immunity. Early studies have found that type I IFNs have the effect of inhibiting tumor cell proliferation and promoting apoptosis. Therefore, type I IFNs are approved for treating clinically specific tumors, including lymphoma, melanoma, renal cell carcinoma (RCC), etc. Recent studies have shown that antigen-presenting cell (APC) and T cell activation mediated by the type I IFN signaling pathway plays a vital role in tumor radiotherapy and chemotherapy. The expression of type I IFNs in tumor tissues, however, is very low. Exogenous type I IFN treatment can not only inhibit the proliferation and survival of tumors, but also activate the anti-tumor immune response, which has thus become an important research focus.

Clinically, high-dose type I IFN treatment will bring better tumor control effects. However, because IFNARs are widely expressed in normal tissues, high-dose type I IFN can induce severe side effects in patients, including flu-like symptoms (fever, headache, etc.), vomit, leukopenia, anemia, thrombocytopenia or the like. In addition, type I IFNs can up-regulate the expression of the immunosuppressive molecule PD-L1, which in turn inhibits the anti-tumor immune response and reduces the therapeutic effect. How to overcome the systemic toxicity and immunosuppression caused by type I IFNs is an important problem to be solved.

### SUMMARY

Despite the presence of TILs, most patients still do not respond to intensive PD-1/PD-L1 therapy. The inventors believe that the defective activation of innate immune APCs may limit the complete activation of tumor-specific T cells after PD-1/PD-L1 blockade. Local delivery of type I IFNs restores the antigen presentation, but also up-regulates PD-L1, which inhibits the subsequent activation of T cells. In order to overcome the limitations and deficiencies existing in the prior art, the present invention provides a fusion protein of IFN-anti-PD-L1 antibody (IFN-anti-PD-L1), which can target both PD-L1 and IFN receptors. It is observed in experiments that the IFN-anti-PD-L1 fusion protein can accumulate in tumor tissues, significantly increase the antigen cross-presentation and overcome the PD-L1-mediated immune suppression. The IFN-anti-PD-L1 fusion protein can simultaneously release immunosuppressive signals and provide costimulatory signals to (re)activate T cells, and can be used as a new generation of anti-PD-L1 antibodies for treating neoplastic diseases.

The objectives of the present invention are achieved by the following technical solutions.

The present invention provides a fusion protein, which is IFN-anti-PD-L1 formed by fusion of IFN and a PD-L1 binding protein. The fusion protein is a homodimer protein or a heterodimer protein.

In the present invention, the homodimer protein includes a first polypeptide and a second polypeptide that are the same, and the first polypeptide and the second polypeptide include an IFN, a PD-L1 binding protein, and an immunoglobulin (Ig) Fc region in sequence from N-terminus to C-terminus.

In the present invention, the heterodimer protein includes a first polypeptide and a second polypeptide that are different, the first polypeptide includes a PD-L1 binding protein, and the second polypeptide includes an Ig Fc region and an IFN located at the N-terminus of the Fc region. An Fc region in the first polypeptide and the Fc region in the second polypeptide are derived from Igs of the same or different subtypes.

In the present invention, the IFN can be selected from a type I IFN, a type I IFN mutant, a type II IFN and/or a type III IFN, such as IFN-α, IFN-β, IFN-γ, IFN-λ1 (IL-29), IFN-λ2 (IL-28a), IFN-λ (IL-28b) and IFN-ω; preferably a type I IFN; more preferably IFN-α4; and further more preferably an IFN-α4 mutant. The IFN can be derived from human or mice. The IFN is preferably IFN-α4 (SEQ ID NO: 13), more preferably an IFN-α4 mutant, and further more preferably mutants mIFN-α4 (L30A) (SEQ ID NO: 25), mIFN-α4 (R144A) (SEQ ID NO: 27), mIFN-α4 (A145G) (SEQ ID NO: 29), mIFN-α4 (R149A) (SEQ ID NO: 31), mIFN-α4 (S152A) (SEQ ID NO: 33), and hIFN-α2 (Q124R) (SEQ ID NO: 35).

In the present invention, the Ig Fc region can be selected from amino acid sequences of constant regions of IgG1, IgG2, IgG3 and/or IgG4, and preferably of IgG1. IgG1 has a stronger ability to induce antibody-dependent cellular cytotoxicity (ADCC) and complement-dependent cytotoxicity (CDC) effects and a longer serum half-life, which is the most common antibody subtype in antibody drugs; and IgG2 and IgG4 have a weaker ability to induce ADCC and CDC effects, but exhibit a longer serum half-life.

In the present invention, the PD-L1 binding protein can be selected from an anti-PD-L1 antibody (an intact antibody), a single-chain variable fragment (ScFv), an Fab fragment, and an F(ab′)₂ fragment; and the anti-PD-L1 antibody is selected from: preferably Tecentriq, Bavencio, Imfinzi, KN035, CS1001, KL-A167, SHR-1316 and/or YW243.55.S70, more preferably ScFv that binds to PD-L1, and further more preferably YW243.55.S70.

In the present invention, the first polypeptide and the second polypeptide of the homodimer preferably include an amino acid sequence shown in SEQ ID NO: 3; and the first polypeptide of the heterodimer preferably includes an amino acid sequence shown in SEQ ID NO: 1, and the second polypeptide includes an amino acid sequence shown in SEQ ID NO: 2, 37, 39, 41, 43, 45, or 47.

The present invention also provides use of the fusion protein, where the IFN-anti-PD-L1 is applied to tumor cells, which can inhibit the growth and/or migration of tumor cells.

The present invention provides use of the IFN-anti-PD-L1 fusion protein in treating neoplastic diseases and use of the IFN-anti-PD-L1 fusion protein in the preparation of drugs or kits. The tumor is preferably a tumor or an advanced tumor against which PD-1/PD-L1 blockade alone is ineffective, and more preferably a tumor that is resistant or irresponsive to an anti-PD-1/PD-L1 antibody alone; and the above tumor is preferably B-cell lymphoma, colon cancer and melanoma.

The present invention provides a pharmaceutical preparation or a pharmaceutical composition, and the active ingredient of the pharmaceutical preparation includes the fusion protein IFN-anti-PD-L1 of the present invention.

The present invention provides a kit including the fusion protein IFN-anti-PD-L1 of the present invention.

The present invention provides nucleic acid molecules encoding the fusion protein IFN-anti-PD-L1 of the present invention.

Among the nucleic acid molecules: 1) nucleic acid molecules encoding the homodimer preferably have nucleotide sequences shown in SEQ ID NO: 6; and
2) nucleic acid molecules encoding the heterodimer can have nucleotide sequences selected from SEQ ID NOS: 4, 5, 7, 8, 9, 10, 38, 40, 42, 44, 46, and 48, and preferably shown in SEQ ID NO: 4 and SEQ ID NO: 5, SEQ ID NO: 4 and SEQ ID NO: 40, or SEQ ID NO: 4 and SEQ ID NO: 42.

The present invention provides a vector including the above nucleic acid molecules.

The present invention provides a cell including the fusion protein IFN-anti-PD-L1 of the present invention or nucleic acid molecules encoding the fusion protein, which is used for producing the fusion protein. The cell is selected from non-human mammalian cells, and preferably from CHO and HEK293 cells.

The present invention provides a method for treating tumors, including administering an effective amount of the fusion protein IFN-anti-PD-L1 to a cancer patient. The tumor is preferably a tumor against which PD-1/PD-L1 blockade alone is ineffective.

The present invention provides a method for treating a tumor or an advanced tumor against which PD-1/PD-L1 blockade alone is ineffective, including administering effective amounts of IFN-α and an anti-PD-L1 antibody to a patient. Moreover, the present invention provides use of IFN-α and an anti-PD-L1 antibody together in the preparation of a pharmaceutical composition, a pharmaceutical preparation or a kit. The present invention also provides a pharmaceutical composition, a pharmaceutical preparation or a kit including IFN-α and an anti-PD-L1 antibody. The IFN-α needs to be intratumourally administered.

On this basis, the present invention provides a combination therapy of an anti-PD-1/PD-L1 antibody and IFN-anti-PD-L1, and a pharmaceutical composition or a kit including the anti-PD-1/PD-L1 antibody and IFN-anti-PD-L1 fusion protein. The therapy includes administering effective amounts of an anti-PD-1/PD-L1 antibody and IFN-anti-PD-L1 to a patient sequentially or simultaneously. The tumor is a tumor or an advanced tumor that is resistant or irresponsive to PD-1/PD-L1 blockade alone; and preferably, a tumor that is resistant or irresponsive to an anti-PD-1/PD-L1 antibody alone. Or, a patient with the tumor suffers from diseases related to defects/disorders in transport of peripheral lymphocytes, and the peripheral lymphocytes of the patient cannot migrate to tumor tissues.

The present invention provides use of the fusion protein IFN-anti-PD-L1 and an anti-PD-1/PD-L1 antibody together in the preparation of a pharmaceutical composition or a kit for treating tumors. The tumor is a tumor or an advanced tumor against which PD-1/PD-L1 blockade alone is ineffective; and preferably, a tumor that is resistant or irresponsive to an anti-PD-1/PD-L1 antibody alone. Or, a patient with the tumor suffers from diseases related to defects/disorders in transport of peripheral lymphocytes, and the peripheral lymphocytes of the patient cannot migrate to tumor tissues.

The present invention provides use of IFN-α and an anti-PD-L1 antibody together in the preparation of a pharmaceutical composition, a pharmaceutical preparation or a kit for treating a tumor or an advanced tumor against which PD-1/PD-L1 blockade alone is ineffective, and the IFN-α needs to be intratumourally administered.

The present invention provides a pharmaceutical composition, a pharmaceutical preparation or a kit including IFN-α and an anti-PD-L1 antibody, and the IFN-α needs to be intratumourally administered.

The present invention provides use of the fusion protein IFN-anti-PD-L1 in up-regulating the expression of IFN receptors in leukocytes; the leukocytes are preferably CD45+ cells; and the IFN receptors are preferably IFNARs. Moreover, the present invention provides use of the fusion protein IFN-anti-PD-L1 in the preparation of a composition for up-regulating the expression of IFN receptors in leukocytes.

The present invention provides use of the fusion protein IFN-anti-PD-L1 in activating DC cells or TIL cells. Moreover, the present invention provides use of the fusion protein IFN-anti-PD-L1 in the preparation of a composition for activating DC cells or TIL cells.

The present invention provides use of the fusion protein IFN-anti-PD-L1 in activating tumor-resident T cells. Moreover, the present invention provides use of the fusion protein IFN-anti-PD-L1 in the preparation of a composition for activating tumor-resident T cells.

The above “use” can mean use for a therapeutic purpose or use for a non-therapeutic purpose.

The present invention provides an IFN-α4 mutant involving mutation that weakens the affinity with a receptor, including mIFN-α4 (L30A) (SEQ ID NO: 25), mIFN-α4 (R144A) (SEQ ID NO: 27), mIFN-α4 (A145G) (SEQ ID NO: 29), mIFN-α4 (R149A) (SEQ ID NO: 31), mIFN-α4 (S152A) (SEQ ID NO: 33), and/or hIFN-α2 (Q124R) (SEQ ID NO: 35).

The present invention provides isolated nucleic acid molecules encoding the IFN-α4 mutant, and the nucleic acid molecules have nucleotide sequences shown in SEQ ID NO: 26, 28, 30, 32, 34, or 36.

The present invention provides use of the IFN-α4 mutant in the preparation of a fusion protein or drug for treating tumors.

### Terms and Definitions

Unless otherwise specified, the terms and definitions used in this application have meanings commonly used in the art and are known to those skilled in the art.

As used in this application, the term “tumor site” refers to an in vivo or in vitro location that includes or is suspected to include tumor cells. The tumor site includes a solid tumor and a location close to or adjacent to where a tumor grows.

As used in this application, the term “administration” refers to systemic and/or topical administration. The term “systemic administration” refers to non-topical administration, so that the administered substance may affect several organs or tissues in the entire body, or the administered substance may traverse several organs or tissues in the entire body to reach a target site. For example, administration to the circulatory system of a subject can cause the expression of a therapeutic product by the administered vector in more than one tissues or organs, or can cause the expression of a therapeutic product by the administered vector at a specific site. For example, this is attributed to natural tropism or operable connection with tissue-specific promoter elements. Those skilled in the art will understand that the systemic administration encompasses various forms of administration, including but not limited to: parenteral administration, intravenous administration, intramuscular administration, subcutaneous administration, transdermal administration, intratumoural administration, oral administration, etc.

The term “topical administration” refers to administration at or around a specific site. Those skilled in the art will understand that topical administration encompasses various forms of administration, such as injection directly into a specific site or injection around the specific site (e.g., intratumoural administration).

As used herein, the term “therapeutically effective amount” refers to an amount of the IFN of the present invention or a component in the kit of the present invention required to achieve the purpose of treating a target disease or condition (e.g., tumor/cancer, for example, for causing the tumor regression or reducing the tumor size). The effective amount can be determined for a specific purpose through practice and in a conventional manner. In particular, the therapeutically effective amount may be an amount required to achieve the following purposes: reducing the number of cancer cells; reducing the tumor size; inhibiting (i.e., slowing or stopping) the infiltration of cancer cells into peripheral organs; inhibiting (i.e., slowing or stopping) the tumor metastasis; and inhibiting the tumor growth; and/or alleviating one or more symptoms related to cancer.

The term “antibody” encompasses, for example, monoclonal antibodies, polyclonal antibodies, ScFv, and antibody fragments (which exhibit the desired biological or immunological activity). In this application, the terms “immunoglobulin” (Ig) and antibody can be used interchangeably. The antibody can specifically target tumor antigens, for example, surface tumor antigens, such as EGFR, CD4, CD8, and Neu.

The “tumor” of the present invention can be selected from B-cell lymphoma, lung cancer, bronchial cancer, colorectal cancer, prostate cancer, breast cancer, pancreatic cancer, gastric cancer, ovarian cancer, bladder cancer, brain or central nervous system (CNS) cancers, peripheral nerves system (PNS) cancers, esophageal cancer, cervical cancer, melanoma, uterine or endometrial cancer, oral or laryngeal cancer, liver cancer, kidney cancer, cholangiocarcinoma, small intestine cancer or appendix cancer, salivary gland cancer, thymic cancer, adrenal cancer, osteosarcoma, chondrosarcoma, lipoma, testicular cancer, and malignant fibrous histiocytoma (MFH).

The “tumor cell” of the present invention can be selected from cells produced by B-cell lymphoma, lung cancer, bronchial cancer, colorectal cancer, prostate cancer, breast cancer, pancreatic cancer, gastric cancer, ovarian cancer, bladder cancer, brain or CNS cancers, PNS cancers, esophageal cancer, cervical cancer, melanoma, uterine or endometrial cancer, oral or laryngeal cancer, liver cancer, kidney cancer, cholangiocarcinoma, small intestine cancer or appendix cancer, salivary gland cancer, thymic cancer, adrenal cancer, osteosarcoma, chondrosarcoma, lipoma, testicular cancer, and MFH.

The “application” or “use” in the present invention can mean use for a purpose of disease treatment or use for a non-therapeutic purpose, such as scientific research.

The present invention has the following beneficial effects:
1. The IFN-anti-PD-L1 fusion protein provided in the present invention targets both PD-L1 and IFN receptors, and data show that the activation of IFN signals in a tumor microenvironment (TME) can enhance the PD-1/PD-L1 therapy against advanced tumors by inducing stronger T cell activation.
2. Data show that, in the IFN-anti-PD-L1 fusion protein provided in the present invention, the anti-PD-L1 antibody (PD-L1 binding protein) can be used to specifically deliver immunomodulatory molecules to tumor tissues with minimal toxicity. The present invention lays a foundation for the development of a novel anti-PD-L1 antibody targeting tumors.
3. The IFN-anti-PD-L1 fusion protein provided in the present invention can lead to multiple feedforward responses targeting PD-L1 blockade and IFN-α receptor (IFNAR) activation in TME, which increases the targeting effect and enhances the response to IFN therapy, thereby realizing the synergistic effect of anti-PD-L1 and IFN to overcome PD-1/PD-L1 therapy and IFN resistance. Experimental data show that IFN-anti-PD-L1 can simultaneously target tumor tissues to activate APCs and block PD-L1 inhibitory signals, and can be used as a new generation of anti-PD-L1 antibodies for treating tumor diseases.
4. The IFN-anti-PD-L1 fusion protein provided in the present invention, when used in combination with PD-1/PD-L1 blockade, completely eliminates most of the PD-1/PD-L1 -resistant tumors and also induces the memory T cell immune response.
5. The fusion protein provided by the present invention has two structures: homodimer and heterodimer, which exhibits high binding affinity to IFN receptors, more effective antiviral activity and excellent tumor targeting in vivo and in vitro, long serum half-life and prominent tumor control in vivo.
6. The fusion protein provided by the present invention includes a mutant IFNα with reduced affinity, which has more specific binding ability to target cells, avoiding the peripheral off-target effect of IFN.

### BRIEF DESCRIPTION OF THE DRAWINGS

FIGS. 1A-1F illustrate that topical administration of type I IFN overcomes the resistance of advanced tumor to PD-1/PD-L1 blockade. FIG. 1A Balb/c mice (n=3) are subcutaneously inoculated with 3×10⁶ A20 cells; and mice carrying early-stage tumors (<50 mm³) are intraperitoneally (i.p.) administered with 200 μg of anti-PD-L1 antibody on days 11 and 15. FIG. 1B Mice (n=4) carrying advanced A20 tumors (>100 mm³) are treated with 200 μg of anti-PD-L1 antibody on days 15 and 19. The tumor growth is measured twice a week. FIG. 1C Mice are treated according to FIG. 1A and FIG. 1B. Three days after the treatment, lymph node (LN)-draining cells are isolated, and then co-cultivated with irradiated or unirradiated A20 cells for two days. The IFNγELISPOT assay is conducted. FIG. 1D A20 tumor-bearing mice (n=5) are treated with 200 μg of anti-PD-L1 antibody on days 11 and 15 (i.p.), and/or intratumourally injected with 25 μg of IFNα-Fc on day 11 (i.t.). FIG. 1E C57BL/6 mice (n=5) are inoculated with 5×10⁵ MC38 cells. The mice are treated with 200 μg of anti-PD-L1 antibody (i.p.) on days 9, 12 and 15, and/or treated with 25 μg of IFNα-Fc on day 9. Survival curves are shown. FIG. 1F A20 tumor-bearing mice (n=5) are treated with 25 μg of IFNα-Fc on day 11 (i.t. or i.v.). The black arrow indicates treatment with IFNα-Fc. Data are shown as mean ±SEM, and involve at least two independent experiments. *, p<0.05; **, p<0.01; and n.s., not significant.

FIGS. 2A-2I illustrate the construction and properties of the IFN-anti-PD-L1 fusion protein. FIG. 2A is a schematic diagram for the IFN-anti-PD-L1 fusion protein in the form of homodimer or heterodimer. ScFv represents a single-chain variable fragment. FIG. 2B and FIG. 2C are flow cytograms illustrating the binding of proteins in IFNAR1^{-/-} A20 cells FIG. 2B and PD-L1^{-/-} A20 cells FIG. 2C. The numbers indicate the mean fluorescence intensity (MFI). FIG. 2D The biological activity of the IFN-anti-PD-L1 protein is determined by the antiviral infection bioassay. Before infected with vesicular stomatitis virus (VSV)-GFP viruses, L929 cells are mixed with each protein and then cultivated overnight. After the cells are further cultivated for 30 h, the percentage of virus-infected cells is determined by flow cytometry (FCM). FIG. 2E and FIG. 2F Balb/c mice (n=5) are inoculated with 3×10⁶ A20 cells. After tumors are established, 20 μg of the corresponding protein is intratumourally injected (e, treating on days 18 and 22) or intravenously injected (f, treating on days 11 and 15), and the tumor size is measured twice a week. FIG. 2G C57BL/6 mice (n=4 to 8) are inoculated with 5×10⁵ MC38 cells. On days 14 and 18, 25 μg of the control or fusion protein is injected intravenously. FIG. 2H and FIG. 2I Mice are injected intravenously with 25 μg of the indicated protein. The protein concentration in tumor tissue FIG. 2H or serum FIG. 2I is measured by ELISA at different time points. Data are shown as mean ±SEM, and involve at least two independent experiments. *, p<0.05; and **, p<0.01.

FIGS. 3A-3F illustrate that the IFN-anti-PD-L1 fusion protein has less toxic and side effects and excellent anti-tumor effect in vivo. FIG. 3A and FIG. 3B 100 μg of IFN-anti-PD-L1 heterodimer or IFN-anti-HBs protein is injected intravenously into tumor-bearing mice on days 0 and 4. Survival curve FIG. 3A and body weight change FIG. 3B are shown. FIG. 3C 100 μg of IFN-anti-PD-L1 heterodimer or IFN-anti-HBs protein is injected intravenously into MC38 tumor-bearing mice. Serum is collected 6 h or 24 h after the injection. The cytokine cytometric bead array (CBA) technology is used to determine cytokine levels in serum. FIG. 3D On days 11 and 15, IFNα-Fc (12.5 μg), anti-PD-L1 antibody (12.5 μg), a mixture of IFNα-Fc and anti-PD-L1 antibody (12.5 μg+12.5 μg), or IFN-anti-PD-L1 fusion protein heterodimer (25 μg) is injected intravenously into A20 tumor-bearing mice (n=5). The tumor size is measured twice a week. FIG. 3E MC38 tumor-bearing mice are treated with 25 μg of the control protein or IFN-anti-PD-L1 heterodimer. Two days later, tumor tissues are collected, and the PD-L1 level is determined by FCM. FMO represents fluorescence minus one. FIG. 3F Mice are treated according to FIG. 3E. Tumor tissues are harvested two days later. The IFNAR levels in CD45-negative and CD45-positive cells are determined by FCM. Data are shown as mean ±SEM, and involve at least two independent experiments. *, p<0.05; **, p<0.01; ***, p<0.001; n.d., not detectable; and n.s., not significant.

FIGS. 4A-4F illustrate that PD-L1 expressed in the host or tumor cells can effectively mediate the anti-tumor effect of the IFN-anti-PD-L1 fusion protein. FIG. 4A PD-L1 expression in WT A20, PD-L1^{-/-} A20, WT MC38, and PD-L1^{-/-} MC38 cells is determined by FCM. FIG. 4B 30 μg of IFN-anti-PD-L1 heterodimer is injected intravenously into WT or PD-L1^{-/-} tumor-bearing mice. Tumor tissues are collected at different time points after the injection. The concentration of the fusion protein is measured by ELISA. FIG. 4C WT or PD-L1^{-/-} A20 tumor-bearing mice (n=4 to 5) are treated with the control Ig or IFN-anti-PD-L1 on days 11 and 15. The tumor growth is measured twice a week. FIG. 4D WT or PD-L^{-/-} MC38 tumor-bearing mice (n=5 to 6) are treated with the control Ig or IFN-anti-PD-L1 on days 8 and 12. FIG. 4E MC38 tumor-bearing mice are treated with IFN-anti-PD-L1. Tumor tissues are collected two days later. The PD-L1 levels in CD45-negative and CD45-positive cells are assessed by FCM. FIG. 4F PD-L1^{-/-} mice (n=4 to 5) are inoculated with MC38 cells. Mice are treated with 25 μg of the control Ig or IFN-anti-PD-L1 on days 14 and 18. The tumor growth is measured twice a week. Data are shown as mean ±SEM, and involve at least two independent experiments. *, p<0.05; **, p<0.01; ***, p<0.001; and n.s., not significant.

FIGS. 5A-5F illustrate that IFNAR expressed by host cells is essential for tumor control. FIG. 5A A20 tumor-bearing mice (n=5) are treated with IFN-anti-PD-L1 on day 11. To block the IFNAR signaling pathway, mice are intratumourally (i.t.) injected with 100 μg of anti-IFNAR blocking antibody on days 11 and 14. FIG. 5B The expression of IFNAR in (B220+) WT or IFNAR^{-/-} A20 tumor cells in vivo is evaluated by FCM. FIG. 5C IFNAR^{-/-} A20 tumor-bearing mice (n=6) are treated with IFN-anti-PD-L1 on days 16 and 19. The tumor growth is measured twice a week. FIG. 5D WT or IFNAR1^{-/-} mice are inoculated with 5×10⁵ MC38 cells. Mice (n=4 to 5) are treated with 25 μg of IFN-anti-PD-L1 on days 10 and 13. FIG. 5E Tumor-bearing mice (n=5 to 6) are treated with IFN-anti-PD-L1 on days 11 and 14. An anti-CD8 antibody for deletion is administered on days 9, 12 and 16. FIG. 5F Two days after the IFN-anti-PD-L1 treatment, MC38 tumor tissues are isolated. The expression of CD86 in tumor-infiltrating DCs (CD11c+MHCII+) is determined by FCM. The left panel is a representative pattern, and the right panel shows MFI. Data are shown as mean ±SEM, and involve at least two independent experiments. *, p<0.05; **, p<0.01; ***, p<0.001; and n.s., not significant.

FIGS. 6A-6G illustrate that PD-1 blockade further ensures that IFN-anti-PD-L1 induces a feedforward anti-tumor response. FIG. 6A A20 tumor-bearing Balb/c mice (n=4 to 5) are treated with 20 μg of IFN-anti-PD-L1 on day 15 and/or with 100 μg of anti-PD-1 antibody on days 14 and 17. The tumor growth is measured twice a week. FIG. 6B B16 tumor-bearing C57BL/6 mice (n=3 to 5) are treated with 25 μg of IFN-anti-PD-L1 on days 11 and 14 and/or with 100 μg of anti-PD-1 antibody on days 12 and 15. FIG. 6C Mice (n=4) whose tumors completely regress after the combination therapy in FIG. 6A are re-inoculated with 2.5×10⁷ A20 cells. Naive mice inoculated with A20 cells are adopted as the control. FIG. 6D Mice (n=4) are treated with the same IFN-anti-PD-L1 and anti-PD-1 in FIG. 6A. To deplete the cells, mice are injected with 200 μg of anti-CD8 antibody one day before the anti-PD-1 antibody treatment. FIG. 6E Mice are treated with IFN-anti-PD-L1 and/or anti-PD-1 as described in FIG. 6A. 12 days after the treatment, tumor-draining lymph nodes (TDLNs) are isolated to prepare single cell suspensions. The cells are co-cultivated with irradiated or unirradiated A20. The IFNγELISPOT assay is conducted. FIG. 6F Tumor-infiltrating DC (CD11c+) and T (CD8+) cells are isolated from A20 tumor-bearing mice, and co-cultivated in the presence of irradiated A20 cells. IFNα or anti-PD-L1 antibody is added to the medium. Three days later, the supernatant is collected, and the IFNγ level is determined by CBA. FIG. 6G A20 tumor-bearing mice (n=5 to 6) are treated with IFN-anti-PD-L1 and/or anti-PD-1 as described in FIG. 6A. FTY720 is administered once every other day from day 14. In order to delete CD8⁺ T cells in the tumor, 30 μg of anti-CD8 antibody is injected into the tumor on days 14 and 17. The black and blue arrows indicate treatment with anti-PD-1 and treatment with IFN-anti-PD-L1, respectively. Data are shown as mean ±SEM, and involve at least two independent experiments. *, p<0.05; **, p<0.01; and ***, p<0.001.

FIGS. 7A-7B illustrate that the IFN-anti-PD-L1 heterodimer specifically targets tumor tissues and induces less toxicity. FIG. 7A 30 μg of IFN-anti-PD-L1 is injected intravenously into MC38 tumor-bearing mice. Tissues are collected on days 1, 3, and 5 after the injection. The concentration of the fusion protein is determined by ELISA. FIG. 7B MC38 tumor-bearing mice are treated as shown in FIG. 3C, and serum is collected 6 h or 24 h after the injection. The cytokine levels in serum are determined by CBA. Data are shown as mean ±SEM and involve two experimental replicates.

FIG. 8 illustrates that IFN-anti-PD-L1 up-regulates the expression of CD80 in tumor-infiltrating DCs. 2 days after the IFN-anti-PD-L1 treatment, MC38 tumor tissues are isolated. FCM is used to detect the expression of CD80 in tumor-infiltrating DCs (CD11c+MHCII+).

FIGS. 9A-9C illustrate that NK and CD4+ T cells exhibit no significant effect in the anti-tumor response mediated by the combination therapy of anti-PD-1 antibody and IFN-anti-PD-L1. A20 tumor-bearing mice (n=4 to 5) are treated as shown in FIG. 6A. FIG. 9A In order to delete NK cells, 20 μg of anti-asialo GM1 antibody is injected intraperitoneally twice a week from day 13. FIG. 9B In order to delete CD4⁺ T cells, 200 μg of anti-CD4 antibody is injected intraperitoneally twice a week from day 13. The tumor growth is measured twice a week. Data are shown as mean ±SEM and involve two experimental replicates. The black and blue arrows indicate treatment with anti-PD-1 and treatment with IFN-anti-PD-L1, respectively. FIG. 9C Mice are treated according to FIG. 6E, and spleens are isolated to prepare single cell suspensions. The cells are co-cultivated with irradiated or unirradiated A20. The IFNγELISPOT assay is conducted.

FIG. 10 illustrates a schematic mode of the anti-tumor effect mediated by an anti-PD-L1 antibody with IFN (IFN-anti-PD-L1). Anti-PD-L1 specifically delivers IFN to tumor tissues (1). IFN-mediated up-regulation of PD-L1 enhances the tumor-specific targeting (2 and 3). The antibody blocks the PD-L1 /PD-1 signaling pathway to release immune braking signals (3). In addition, IFN-anti-PD-L1 up-regulates the expression of IFNAR (4), which makes tumors more sensitive to treatment (5). In summary, these factors lead to the (re)activation of T cell responses to control the tumor growth (6).

FIG. 11 illustrates the construction of IFNα mutants with reduced affinity. Single site-directed selective mutations are conducted for key sites of type I IFN that interact with the receptors.

FIG. 12 illustrates the activity assay for IFNα-Fc mutants. The biological activity is assayed for IFNα-Fc mutants by the antiviral infection bioassay. Before infected with VSV-GFP viruses, L929 cells are mixed with each protein and then cultivated overnight. After the cells are further cultivated for 30 h, the percentage of virus-infected cells is determined by FCM, and the inhibition rates and EC₅₀ values of different concentrations of protein on cell infection are calculated. The two mutants R144A and A145G have the weakest activity and are potential preferred targets.

FIG. 13 illustrates the in vitro targeting test of mutant IFN-anti-PD-L1 WT PD-L1⁺ A20 cells and PD-L1^{-/-} A20 cells previously subjected to knockout by the CRISPR-Cas9 technology in the laboratory are adopted, different concentrations of bispecific protein are added, and resulting mixtures are incubated for 72 h. The proliferation is detected by a CCK8 kit for cells treated with different concentrations of protein. The ratio of EC₅₀ in PD-L1⁻ A20 cells to EC₅₀ in PD-L1⁺ A20 cells can reflect the targetability of this bispecific protein. It can be found from the normalization of the ratio with the data of wt-mIFNa4-Fc that R144A and A145G are the two with the optimal targetability.

### DETAILED DESCRIPTION OF THE EMBODIMENTS

The present invention is further described in detail through the following examples, but it should be understood that the present invention is not limited by the following content.

### Materials and Methods

### Mice

Female (6 to 8 weeks old) BALB/c mice and C57BL/6 mice were purchased from Beijing Vital River Laboratory Animal Technology Co., Ltd. (Beijing, China). All mice were kept under specific pathogen-free (SPF) conditions in the animal room of the Institute of Biophysics, Chinese Academy of Sciences. Animal care and experiments were conducted in accordance with the guidelines of the Institute of Biophysics, Chinese Academy of Sciences, which followed the protocol approved by Institutional Laboratory Animal Care and Use Committee (IACUC). PD-L1^{-/-} and IFNAR1^{-/-} mice were kept under SPF conditions at UT Southwestern Medical Center. The animal protocol complied with NIH guidelines. The study was approved by the Animal Care and Use Committee of UT Southwestern Medical Center.

### Cell Lines and Reagents

293F cells were provided by Dr. Xu Ting (Alphamab Oncology, Suzhou, Jiangsu Province, China) and were cultivated in SMM 293-TI medium (M293TI, Sino Biological). A20, MC38 and L929 cell lines were purchased from the American Type Culture Collection (ATCC) (Manassas, VA). The anti-PD-1 blocking antibody (4H2) was purchased from Bristol-Myers Squibb (Redwood City, CA). The anti-PD-L1 antibody (10F.9G2) and anti-IFNARI antibody (MAR1-5A3) were purchased from BioXCell (West Lebanon, NH). The anti-CD8 (TIB210) and anti-CD4 (GK1.5) antibodies for deletion were prepared by the laboratory itself. The anti-asialo GM1 antibody was purchased from Biolegend (San Diego, CA).

### Construction of IFN-anti-PD-L1 Fusion Protein

Heterodimer: The light-chain and heavy-chain variable regions of the PD-L1 binding protein (YW243.55.S70) sequence were synthesized according to Patent U.S. Pat. No. 8,217,149B2. The light-chain and the heavy-chain sequences were linked via the GGGGSGGGGSGGGGS linker, and human IgG1Fc (SEQ ID NO: 15) was inserted into the C-terminus of the heavy chain to give a first polypeptide of the heterodimer, named ScFv (PD-L1)-Fc (SEQ ID NO: 1). Then the coding nucleic acid sequence (SEQ ID NO: 4) of ScFv (PD-L1)-Fc was cloned into a pEE12.4 vector (Lonza). The cDNA sequence (SEQ ID NO: 14) of murine IFN-α4 (SEQ ID NO: 13) was cloned and inserted into the N-terminus of human IgG1Fc through the (G4S)₄ linker to give a second polypeptide of the heterodimer, mIFNα4-Fc (SEQ ID NO: 2). The nucleotide sequence (SEQ ID NO: 5) encoding mIFNα4-Fc was cloned into a pEE6.4 vector (Lonza). The heterodimerization of PD-L1 binding protein and IFNα was conducted using the knob-to-holes technique previously reported. Plasmids were transiently transfected into 293F cells at a ratio of 1:2. The supernatant was collected on day 7 after the transfection. The fusion protein was purified using a Protein A-Sepharose column according to the operational manual (Repligen).

During the preparation of the heterodimer, different type I IFNs were also used to prepare the second polypeptides mIFNb-Fc (with an encoding nucleic acid sequence shown in SEQ ID NO: 7), hIFNα2-Fc (with an encoding nucleic acid sequence shown in SEQ ID NO: 8), hIFNb-Fc (with an encoding nucleic acid sequence shown in SEQ ID NO: 9), and mIFNγ-Fc (with an encoding nucleic acid sequence shown in SEQ ID NO: 10), and the obtained heterodimers showed a relatively-high inhibitory activity on tumor cell proliferation. In the above-mentioned heterodimer fusion proteins, IFNα-Fc exhibited a better effect. Moreover, the heterodimer fusion protein composed of mIFNα4-Fc and Anti-PD-L1 exhibited the optimal effect, and relevant comparative data are not described here in detail.

Homodimer: The C-terminus of murine IFN-α4 was linked to the N-terminus of ScFv(PD-L1)-Fc to give a first and second polypeptides of the homodimer fusion protein (SEQ ID NO: 3). The nucleotide sequences (SEQ ID NO: 6) of the polypeptides were cloned into a pEE12.4 vector, and after transfection, the homodimer protein was formed spontaneously through Fc dimerization.

### Flow Cytometry (FCM)

The binding of the fusion protein was detected using PE-anti-human IgG Fc (eBioscience). Specific antibodies: anti-PD-L1 antibody (10F.9G2), anti-IFNAR1 antibody (MAR1-5A3), anti-CD45 antibody (30-F11), anti-CD80 antibody (16-10A1), and anti-CD86 antibody (GL1) were from BioLegend or eBioscience. Cells were suspended in FACS buffer (1% bovine serum albumin (BSA) and 0.05% NaN₃), blocked with an anti-CD16/32 antibody (anti-FcγIII/II receptor, clone 2.4G2) for 30 min, and then stained with a specific antibody for 30 min on ice. The samples were assayed on FACSCalibur or Fortessa flowcytometer (BD Biosciences). Data were analyzed using FlowJo software (TreeStar).

### Antiviral Activity of IFNα

L929 mouse fibroblasts sensitive to VSV infection were used to quantify the biological activity of IFN. The cells were incubated with serially-diluted IFNα-Fc or IFN-anti-PD-L1 at 37° C. overnight. The next day, the cells were infected with VSV-GFP with MOI=5, and then further cultivated for 30 h. The cells were then collected and fixed with 4% PFA. Data were acquired using FACSFotassa flow cytometer (BD Biosciences) and analyzed using FlowJo software (TreeStar). GFP-positive cells were defined as virus-infected cells.

### Quantitative Study of Protein Distribution In Vivo

A20 cells (3×10⁶) were injected subcutaneously (s.c.) into the right side of Balb/c mice. On day 15, mice were injected intravenously with 30 μg of IFNα-Fc or IFN-anti-PD-L1. 3 days after perfusion, different mouse tissues were collected, and the levels of human Fc in the homogenate extracts of different organs and tissues were determined by ELISA.

### Growth and Treatment of Tumors

A20 cells (3×10⁶) were injected subcutaneously into the right side of Balb/c mice. 20 μg of IFN-anti-PD-L1 was then injected intravenously. In order to block PD-1 signaling, one day before IFN-anti-PD-L1 treatment, mice were treated intravenously with 100 μg of anti-PD-1 antibody (4H2) once every two weeks. In order to delete CD8+ T cells, one day before IFN-anti-PD-L1 treatment, 200 μg of anti-CD8 antibody (TIB210) was injected intraperitoneally. In order to block type I IFN signaling, one day before IFN-anti-PD-L1 treatment, 100 μg of anti-IFNAR1 antibody (MAR1-5A3) was injected. C57BL/6 mice were injected subcutaneously with 5×10⁵ MC38 cells at the right side. Mice were then injected intravenously with 25 μg of IFN-anti-PD-L1 twice. The tumor volume was measured twice a week and calculated by (length×width×height/2). In order to block lymphocyte transport, mice were injected intraperitoneally with 25 μg of FTY720 and then 20 μg of FTY720 was given every other day to maintain the blocking.

### Detection of Tumor Antigen-specific T Cells by ELISPOT

Lymph nodes (LNs) or spleens were isolated from tumor-bearing mice to prepare single cell suspensions. A single dose of 60 Gys (10 Gys/min, 6 min) was used to irradiate A20 tumor cells. Spleen cells or LN cells were co-cultivated with irradiated tumor cells for 48 h at a ratio of 4:1. According to the operational plan of the manufacturer (BD Biosciences), IFN-γ production was measured with an IFN-γELISPOT assay kit. Cytokine spots were assayed with an immunospot analyzer (CTL).

### In Vitro Cultivation and Functional Analysis

A tumor tissue was collected, cut into small pieces, and suspended in a digestion buffer (RPMI-1640 medium with 1 mg/mL collagenase IV and 100 μg/mL DNase I). After digestion at 37° C. for 45 min, a single cell suspension was prepared by a 70 μm cell strainer, and CD8+ T cells and DCs (MHCII+CD11c+) were sorted by FACS. T cells, DCs and irradiated tumor cells were co-cultivated in the presence of IFNα (2 ng/mL) or anti-PD-L1 antibody (10 μg/mL) at a ratio of 10:1:2.5. Three days later, the supernatant was collected, and the IFN-γ level was determined by CBA.

### Statistical Analysis

Data were shown as mean ±SEM. Unpaired Student's two-tailed t test was used for comparative statistical analysis. GraphPad Prism version 5.0 (GraphPad Software) was used for analysis. *, ** and *** were used to indicate the statistically significant differences of p<0.05, p <0.01 and p<0.001, respectively.

### Example 1: Topical Administration of Type I IFN Overcomes the Resistance of Tumor to PD-1/PD-L1 Blockade Therapy

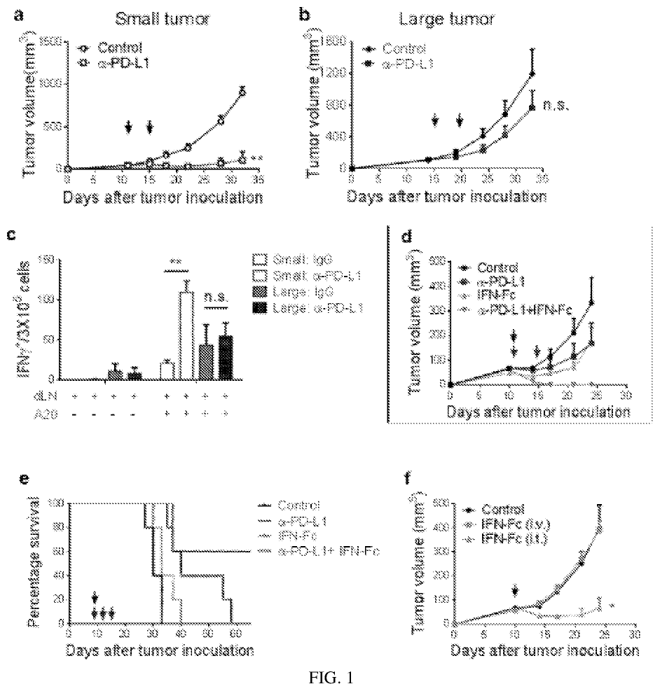

A recent study showed that the clinical response of a patient to immune checkpoint blockade was related to the T cell activation and tumor burden. Consistent with that, the present invention found that the anti-PD-L1 antibody exhibited effective tumor control in small A20 tumors (<50 mm³) (FIG. 1A). On the contrary, when the tumor became larger (>100 mm³), the anti-tumor effect was significantly reduced (FIG. 1B). Advanced tumors may have formed a variety of mechanisms to suppress the anti-tumor immune response. In fact, when comparing the T cell activation in small tumors with the T cell activation in large tumors, it was observed that PD-L1 blockade induced strong T cell activation in small tumors, and the same treatment resulted in limited effect on T cells in advanced tumors (FIG. 1C). The data implied that the insufficient T cell activation may be caused by ineffective response of advanced tumors to immune checkpoint blockade.

In order to verify this hypothesis, experiments were designed to explore whether providing type I IFN (a powerful cytokine that enhances cross-presentation to cytotoxic T cells) can improve the PD-1/PD-L1 blockade therapy. Advanced tumors were treated with the combination therapy of PD-L1 blockade and IFNα. Neither anti-PD-L1 antibody nor IFNα (IFNα4) alone was unable to control tumors, and all tumors eventually progressed (FIG. 1D). Impressively, the combination therapy induced a stronger anti-tumor effect, leading to complete tumor elimination in all treated mice (FIG. 1D). A similar synergistic effect was found in another tumor model MC38 (FIG. 1E). Type I IFN signals acted locally in TME, because intratumoural delivery of IFNα effectively controlled the tumor growth (FIG. 1F). In contrast, when IFN was delivered systemically, these effects disappeared completely.

In summary, the above data indicate that type I IFN and PD-1/PD-L1 blockade have a synergistic effect and can control advanced tumors. This also indicates that IFN needs to target TME to achieve optimized anti-tumor effects.

### Example 2: Construction of IFN-anti-PD-L1 Fusion Protein for Specific Delivery of IFN to Tumor Tissues

Topical administration of IFN to tumors cannot be implemented for most patients. In addition, systemic delivery of type I IFN usually leads to limited anti-tumor activity and severe side effects. Targeting antibodies with cytokines have proven to be an effective strategy for topical delivery of immunomodulatory molecules. However, it is quite difficult to identify tumor-specific molecules for therapeutic targeting. It has been reported that PD-L1 is highly expressed in tumor tissues. Recent studies have shown that anti-PD-L1 antibodies specifically accumulate in PD-L1 -positive tumor tissues. Moreover, in addition to the anti-tumor function, IFNs can powerfully induce the expression of PD-L1, thus inhibiting the response of T cells to tumors. In order to overcome this counteracting effect and realize the mutual promotion for immune (re)activation in TME, the present invention proposes to use an IFN and anti-PD-L1 antibody to construct a fusion protein IFN-anti-PD-L1, which can further up-regulate the expression of PD-L1 in tumor tissues, thereby leading to increased accumulation of antibodies.

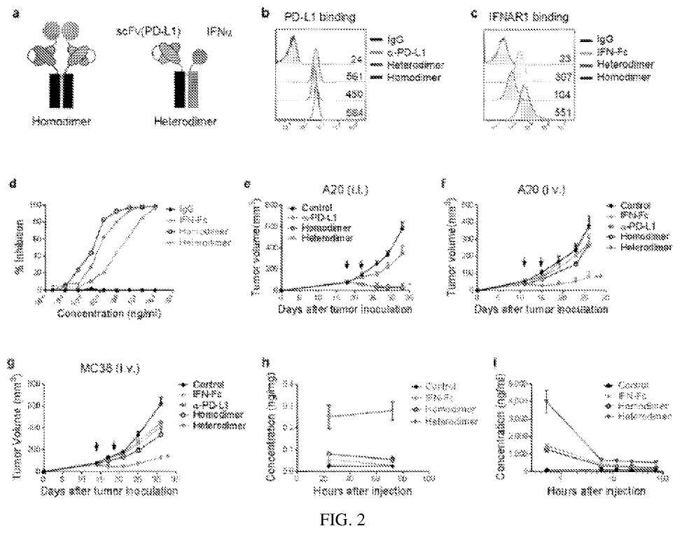

In the present invention, in order to verify this hypothesis, a fusion protein with anti-PD-L1 antibody scFv [scFv (PD-L1)] and IFNα was produced in the form of homodimer or heterodimer (FIG. 2A). In order to evaluate the obtained IFN-anti-PD-L1 fusion protein (using IFN-α4), its affinity with PD-L1 or IFN-α receptor 1 (IFNAR1) was determined. A20 cells were positive for both PD-L1 and IFN receptors. Therefore, one receptor was knocked out from A20 cells, and the binding of the fusion protein to another receptor was determined. In IFNAR1^{-/-} A20 cells expressing PD-L1, the fusion protein exhibited similar affinity to anti-PD-L1 antibodies (FIG. 2B). In PD-L1^{-/-} A20 cells expressing IFNAR, the heterodimer exhibited reduced binding compared with IFN-Fc or the homodimer (FIG. 2C).

TABLE 1
Protein binding data of the Example 2.

| | | | | IFN-anti- | IFN-anti- |
|---|---|---|---|---|---|
| | IgG | | | PD-L1 | PD-L1 |
| Group | (control) | Anti-PD-L1 | IFN-Fc | heterodimer | homodimer |
| IFNAR1^{-/-} | − | ++ | − | ++ | ++ |
| A20 | | | | | |
| PD-L1^{-/-} | − | − | ++ | + | ++ |
| A20 | | | | | |

Notes:
“+” means binding strength,
“−” means no binding,
and blank space means no experiment.

The fusion protein effectively protected L929 cells from VSV infection, indicating that the antiviral activity of IFN remained unchanged (FIG. 2D).

In summary, these data indicate that the fusion protein IFN-anti-PD-L1 in the form of homodimer or heterodimer can bind to PD-L1 while remaining the effective biological activity of IFN.

### Example 3: Targeted Delivery of IFN Through Anti-PD-L1 to Control Advanced Tumors

In view of the potent activity of IFN-anti-PD-L1 (IFN-α4) fusion protein in vitro, further studies were conducted to explore whether the fusion protein can control the tumor growth in vivo. Mice with advanced A20 tumors were treated with the fusion protein (i.t.). Although anti-PD-L1 antibodies failed to control the tumor growth, the IFN-anti-PD-L1 fusion protein in the form of homodimer or heterodimer overcame the anti-PD-L1 resistance and induced complete tumor regression (2e) in most treated mice.

In order to test the targeting effect of the fusion protein, A20 tumor-bearing mice were systematically treated with the fusion protein. Surprisingly, although the homodimer exhibited a higher binding affinity to IFN receptors and a higher antiviral activity in vitro (FIG. 2C and FIG. 2D), only the heterodimer instead of the homodimer could effectively inhibit the tumor growth (FIG. 2F) when delivered in vivo. A similar effect was observed in the MC38 model (FIG. 2G). We want to know whether this difference is due to the different dynamics in vivo. Further experimental results showed that compared with the homodimer, the heterodimer had a higher accumulation level in tumor tissues (FIG. 2H). In addition, the serum half-life of the heterodimer increased significantly (FIG. 2I). These data also indicate that the heterodimer has a better anti-tumor effect, and also mean that the heterodimer is a preferred candidate for in vivo studies.

In summary, these data show that targeted delivery of IFN by anti-PD-L1 antibodies can induce effective anti-tumor effects, resulting in improved tumor control.

### Example 4: Tumor-targeted IFN-anti-PD-L1 Shows Less Toxicity and Strong Anti-tumor Activity

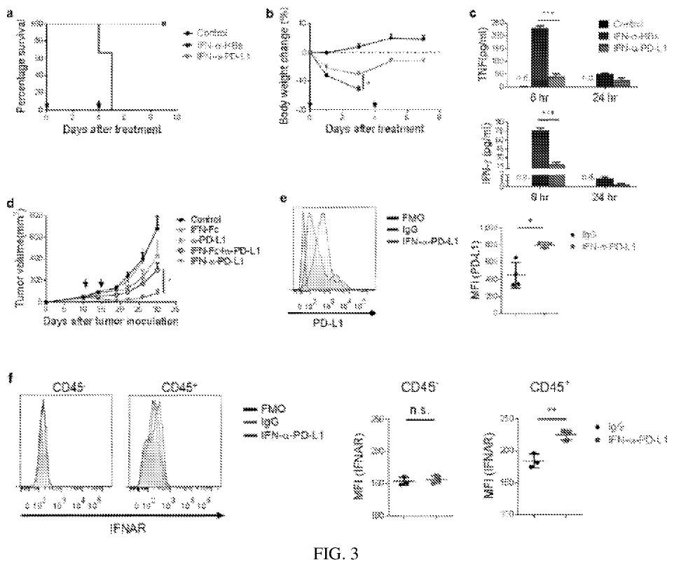

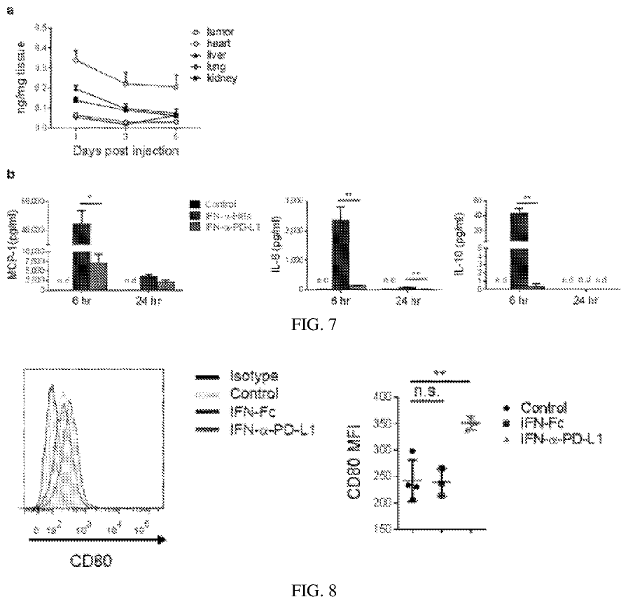

The application of type I IFN is limited due to severe side effects during systemic delivery. In order to test the in vivo toxicity of anti-PD-L1 antibodies with IFN (IFN-α4), the heterodimer (IFN-anti-PD-L1) or non-targeting control IFN-anti-HBs (anti-Hepatitis B virus (HBV) surface protein) fusion protein was adopted at a high dose. After the second injection of IFN-anti-HBS, tumor-bearing mice experienced severe weight loss, activity reduction and fur wrinkling, and all died within one day (FIG. 3A). In contrast, none of the mice treated with IFN-anti-PD-L1 died, and these mice recovered after mild weight loss (FIG. 3A and FIG. 3B). The IFN-anti-PD-L1 fusion protein accumulated in tumors but not in normal tissues (FIG. 7A).

In order to better evaluate the side effects, the cytokine level in serum was determined after the first injection. Impressively, non-targeting IFN-anti-HBs induced high expression of inflammatory cytokines TNF, IFN, MCP-1, IL-6, IL-10 and the like (FIG. 3C and FIG. 7B).

Tumor-specific targeting is essential for the anti-tumor effect of the fusion protein because a simple mixture of IFN-Fc and an anti-PD-L1 antibody does not produce a synergistic effect like that generated by the IFN-anti-PD-L1 fusion protein (FIG. 3D).

In summary, these data show that IFN-anti-PD-L1 can target tumor tissues to inhibit the tumor growth, and has less toxicity and side effects.

### Example 5: IFN-anti-PD-L1 Up-regulates PD-L1 and IFNAR Receptors in TME

Since type I IFN is the most effective cytokine for inducing PD-L1 expression, the PD-L1 level in TME was determined after systemic treatment with IFN-anti-PD-L1 (IFN-α4 heterodimer). IFN-anti-PD-L1 significantly increased the expression of PD-L1 in tumor tissues (FIG. 3E). Increased PD-L1 expression could enhance the tumor-specific accumulation of the fusion protein. Interestingly, the level of IFN receptors in CD45⁺ cells (leukocytes) also increased (FIG. 3F), making the cells more sensitive to IFN treatment. In summary, data show that IFN-anti-PD-L1 leads to multiple feedforward responses, which may further enhance tumor-targeted and anti-tumor effects.

### Example 6: PD-L1 in Tumor Cells is not Necessary for the Anti-tumor Effect of the IFN Fusion Protein

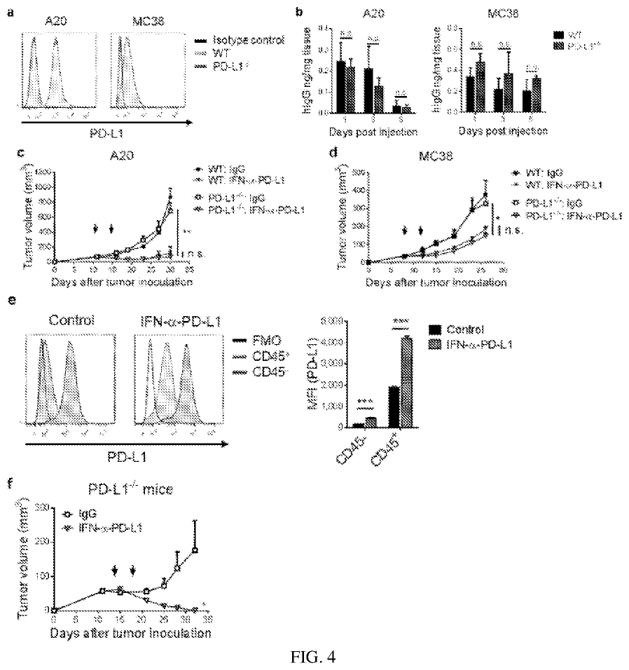

Many tumor cells overexpress PD-L1 as a strategy to evade immune response. PD-L1 can be further induced by inflammatory cytokines in many cells outside tumors. Whether PD-1/PD-L1 blockade acts on PD-L1 expressed by tumor cells or acts on PD-L1 expressed by non-tumor cells has not yet been settled. In order to determine whether PD-L1 in tumor cells is necessary for anti-PD-L1 antibodies with IFN, the present invention used the CRISPR/Cas9 technology to knock out PD-L1 from tumor cells. PD-L1 expression was completely eliminated in the A20 and MC38 tumor cells subjected to knockout (FIG. 4A). IFNs could induce the expression of PD-L1. When stimulated by IFNα, PD-L1 was up-regulated in wild-type (WT) cells, but the cells subjected to knockout still did not express PD-L1 (data were not shown).

To test whether PD-L1 on tumor cells is necessary for targeting, the fusion protein IFN-anti-PD-L1 (IFN-α4 heterodimer) was used to treat WT or PD-L1 -knockout (PD-L1^{-/-}) tumor-bearing mice. Protein levels in tumor tissues were measured. To our surprise, the fusion protein accumulated in tumor tissues regardless of whether the tumor cells express PD-L1 (FIG. 4B). When using IFN-anti-PD-L1 to treat tumors, both PD-L1 -knockout tumors and WT tumors were able to be effectively controlled (FIG. 4C and FIG. 4D). These data indicated that PD-L1 in tumor cells was not necessary for the anti-tumor effect.

The previous data showed that IFN-anti-PD-L1 created a feedforward loop to up-regulate the expression of PD-L1 in TME (FIG. 3E). Since both tumor and stromal cells can express PD-L1, the expression levels of PD-L1 in different cell subgroups in tumor tissues were detected. IFN-anti-PD-L1 treatment significantly up-regulated PD-L1 expression in tumor and stromal cells (FIG. 4E). Since PD-L1 in tumor cells is not necessary, we want to know whether PD-L1 is necessary in host cells. Interestingly, IFN-anti-PD-L1 well controlled the tumor growth in PD-L1 -deficient mice (FIG. 4F). In summary, these data indicate that PD-L1 expressed in host cells or tumor cells is sufficient to mediate the tumor-targeted and anti-tumor effects of the fusion protein.

### Example 7: IFN-anti-PD-L1 Fusion Protein Promotes the Activation of APC and T Cells in TME

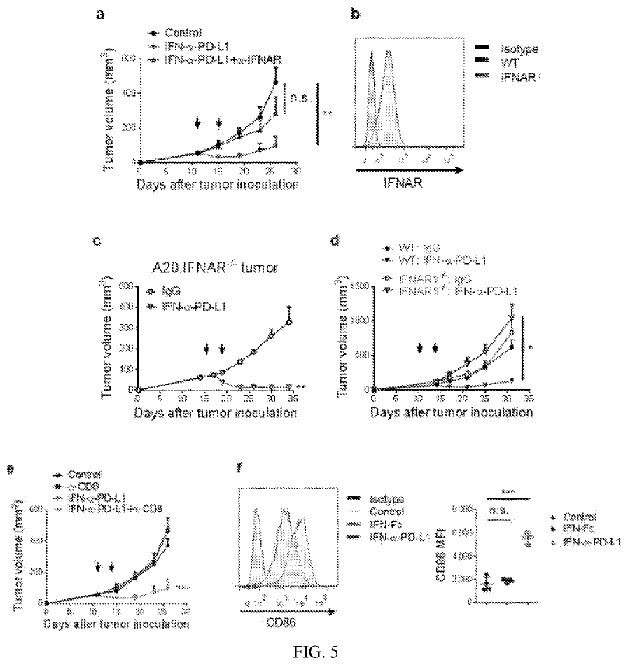

Next, it was investigated whether type I IFN signaling is necessary for anti-tumor effects. Mice were treated with an anti-IFNAR blocking antibody during treatment with the IFN-anti-PD-L1 fusion protein (heterodimer). The anti-IFNAR antibody completely eliminated the anti-tumor effect of the fusion protein, suggesting the important role of type I IFN signaling (FIG. 5A).

IFNAR was expressed in both tumor and host cells. To detect whether IFN receptors in tumor cells are necessary, IFNAR1 was knocked out from tumor cells (FIG. 5B). Interestingly, IFN-anti-PD-L1 effectively controlled the tumor growth in mice carrying A20.IFNAR1^{-/-} tumors (FIG. 5C).

Since IFN receptors in tumor cells are not necessary, we investigated whether the receptors expressed on host cells are necessary. MC38 tumors were inoculated into WT or IFNAR^{-/-} mice, and then the mice were treated with the fusion protein. It was observed in the experiment that the anti-tumor effect disappeared in deficient mice, suggesting that IFN receptors played a more important role in the host (FIG. 5D).

CD8+ T cells are necessary for anti-tumor effects. When CD8+ T cells are blocked with an anti-CD8 antibody, the anti-tumor effect is completely eliminated (FIG. 5E). Previous studies have shown that type I IFN can enhance DC cross-presentation in TME, leading to better T cell activation. In fact, IFN-anti-PD-L1 treatment increased the expression of CD86 and CD80, which are marker molecules for DC activation (FIG. 5F and FIG. 8). As a control, no significant activation of DC was observed in tumors treated with IFN-Fc, indicating the important role of tumor-specific targeting.

In summary, these data indicate that the IFN fusion protein mediates the anti-tumor effect mainly through IFN signaling in host cells.

### Example 8: Targeted Delivery of IFN Through an Anti-PD-L1 Antibody Overcomes the Resistance of Tumors to PD-1 Blocking Therapy

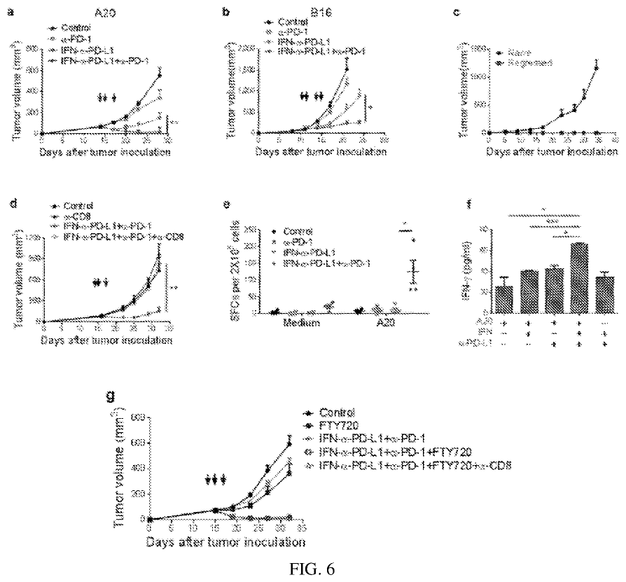

Advanced tumors were often resistant to PD-1/PD-L1 blocking therapy. In fact, neither anti-PD-1 antibody therapy nor anti-PD-L1 antibody therapy could control the growth of advanced A20 tumors (FIG. 6A). Compared with PD-1/PD-L1 blockade, the IFN-anti-PD-L1 fusion protein (IFN-α4 heterodimer) showed a better anti-tumor effect. However, some tumors eventually relapsed after being initially controlled (FIG. 2F to FIG. 2G). The overexpression of PD-L1 after immunotherapy may further limit the T cell-mediated tumor control. Therefore, it was assumed that the combination therapy of anti-PD-1 antibody and IFN-anti-PD-L1 can overcome the resistance of tumors to IFN or PD-1/PD-L1 blocking therapy. In fact, the combination therapy resulted in better tumor control, and advanced tumors almost completely regressed (FIG. 6A).

B16F10 melanoma is a well-known mouse tumor model that is resistant to PD-1/PD-L1 blocking therapy. Consistent with previous reports, PD-1/PD-L1 blockade exhibited no effect on tumor growth in the B16F10 model (FIG. 6B). IFN-anti-PD-L1 treatment only partially controlled the tumor. Interestingly, the combination therapy of IFN-anti-PD-L1 and PD-1 blockade significantly improved the anti-tumor effect.

To test whether the anti-tumor response mediated by IFN-anti-PD-L1 leads to prolonged protective T cell immunity, a lethal dose of A20 cells was again inoculated into mice that experienced complete tumor regression after the combination therapy. All mice resisted the re-attack of tumor cells, confirming that the fusion protein induced a memory adaptive immune response (FIG. 6C).

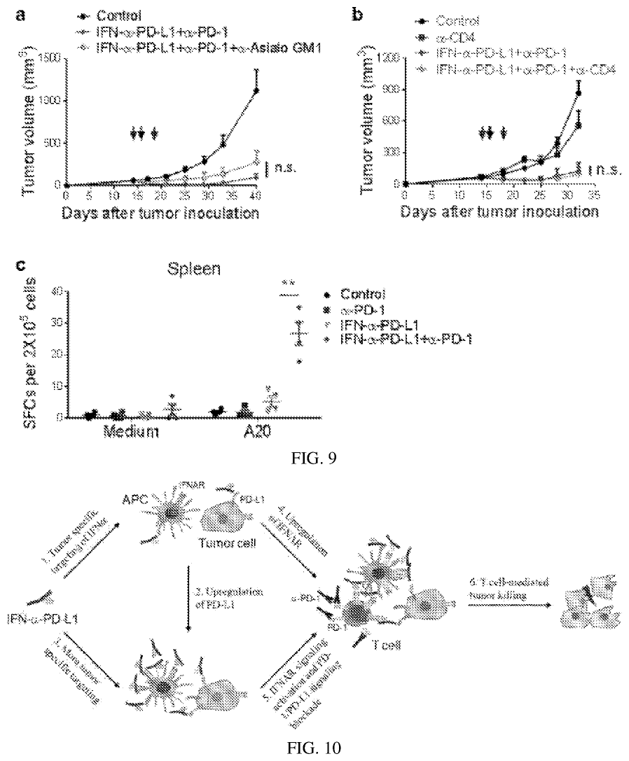

To determine which cell population or populations are required for the combination therapy, mice that experienced the combination therapy were treated with an anti-NK, CD4+ or CD8+ T cell antibody for deletion. In the absence of CD8+ T cells, the anti-tumor effect disappeared completely (FIG. 6D). In contrast, the deletion of NK or CD4+ T cells resulted in a limited effect (FIG. 9A and FIG. 9B). In order to detect whether tumor-specific T cells are produced after the treatment, cells were isolated from LN or spleen tissues and then co-cultivated with irradiated A20 tumor cells. The IFN-αELISPOT assay was conducted to assess the tumor-specific T cell response. PD-1/PD-L1 blockade alone showed a limited effect on T cell activation (FIG. 6E and FIG. 9C). In contrast, IFN-anti-PD-L1 (IFN-α4 heterodimer) induced a better response. Importantly, the combination therapy of IFN-anti-PD-L1 and PD-1 blockade significantly increased the number of tumor-specific T cells (FIG. 6E and FIG. 9C).

To test IFN, PD-L1 and tumor cells in an established in vitro system, TME could be better reproduced, and DC and T cells were isolated from tumors established in vivo. Cells were cultivated for three days in the presence of IFN, anti-PD-L1 antibody, or a combination of the two. Although monotherapy showed a limited effect, the combination therapy of IFN and anti-PD-L1 antibody significantly increased the production of IFNγ by T cells (FIG. 6F).

In summary, these data indicate that the combination of IFN and PD-1/PD-L1 blockade coordinately induces a strong tumor-specific T cell response, which can overcome the resistance of tumors to immune checkpoint blockade in advanced tumors.

### Example 9: Targeted Delivery of IFN Activates Tumor-resident T Cells for Tumor Control

Data show that tumor-specific T cells play an important role in the anti-tumor immune response. These T cells may mainly come from two sources. Some are T cells that are already present in the tumor tissue, while others are newly-activated T cells that migrate from the periphery to the tumor tissue. IFN can not only stimulate DC to activate TIL, but also increase chemokines that attract T cells. To test which T cell populations are necessary, FTY720 was used to block the transport of peripheral lymphocytes to tumor tissues. Interestingly, even when lymphocyte transport was blocked, similar to the control group, the combination treatment with IFN and PD-1/PD-L1 blockade could also control tumors (FIG. 6G). These data showed that reactivation of pre-existing T cells by IFN-anti-PD-L1 (IFN-α4 heterodimer) was sufficient for tumor control. Consistently, the topical deletion of CD8⁺ T cells reduced all anti-tumor effects (FIG. 6G). In summary, these data indicate that PD-L1 blockade can reverse the T-cell depletion state, and IFN effectively reactivates the partially-recovered resident T cells for tumor control.

### Example 10: Construction of IFNα Mutants With Reduced Affinity

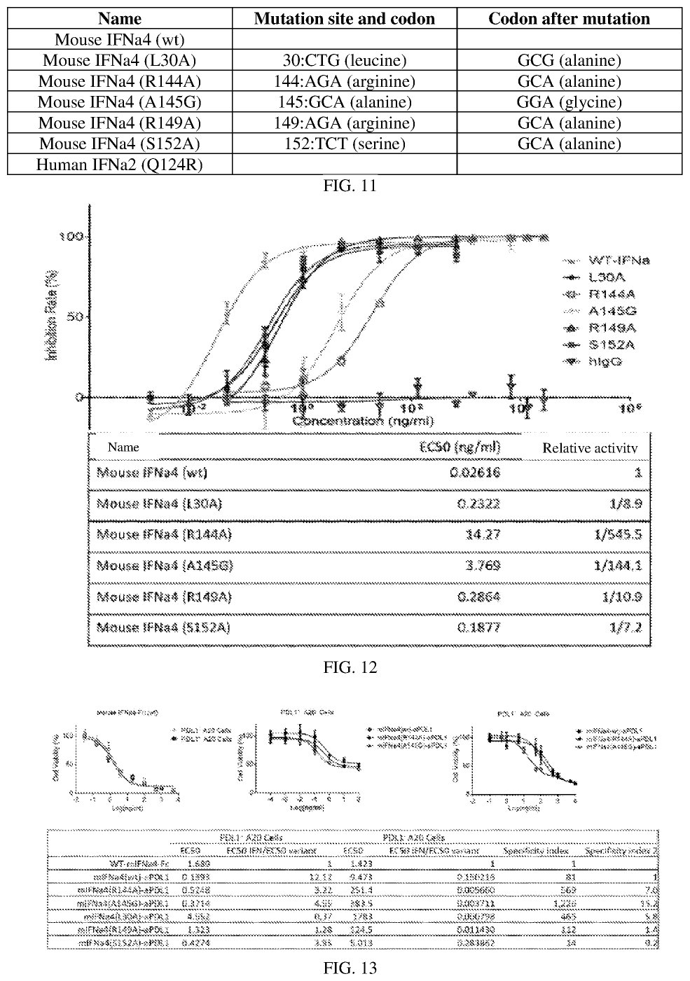

Site-directed mutations were conducted at the known key amino acid sites of IFNα that bind to the receptor IFNAR. Specific mutation sites were shown in FIG. 11. IFNα mutants with reduced affinity were screened. The amino acid sequences and coding nucleic acid sequences of the IFNα mutants were shown in SEQ ID NOS: 25 to 36 in the sequence list of the specification.

TABLE 2

| | Amino acid | Coding nucleic acid |
|---|---|---|
| Name | sequence | sequence |
| Mouse IFNα4 (L30A) | SEQ ID NO: 25 | SEQ ID NO: 26 |
| Mouse IFNα4 (R144 A) | SEQ ID NO: 27 | SEQ ID NO: 28 |
| Mouse IFNα4 (A1456) | SEQ ID NO: 29 | SEQ ID NO: 30 |
| Mouse IFNα4 (R149A) | SEQ ID NO: 31 | SEQ ID NO: 32 |
| Mouse IFNα4 (S152A) | SEQ ID NO: 33 | SEQ ID NO: 34 |
| human IFNα2 (Q124R) | SEQ ID NO: 35 | SEQ ID NO: 36 |

### Example 11: Activity Assay for IFNα-Fc Mutants

The biological activity was assayed for IFNα-Fc mutants by the antiviral infection bioassay. Before infected with VSV-GFP viruses, L929 cells were mixed with each protein and then cultivated overnight. After the cells were cultivated for 30 h, the percentage of virus-infected cells was determined by FCM, and the inhibition rates and EC₅₀ values of different concentrations of protein on cell infection were calculated (FIG. 12). Results show that the constructed mutants all exhibit a certain decrease in activity, and the two mutants R144A and A145G have the weakest activity and are potential preferred targets.

### Example 12: In vitro Targeting Test of Mutant IFN-anti-PD-L1

WT PD-L1⁺ A20 cells and PD-L1^{-/-} A20 cells were adopted, and the proliferation was detected by a CCK8 kit for cells treated with different concentrations of protein. The results showed that, compared with the WT fusion protein, the mutant IFN-anti-PD-L1 exhibited a lower EC₅₀ value for inhibiting the proliferation of targeting-deficient PD-L1^{-/-} A20 cells. On PD-L1^{+/+} A20 cells with targetability, the mutant IFN-anti-PD-L1 and WT fusion protein exhibited no significant difference in terms of EC₅₀ for the inhibition on cell proliferation (FIG. 13). In the mutant IFN-anti-PD-L1 fusion protein, the first polypeptide, Anti-PD-L1 (ScFv(PD-L1)-Fc), had an amino acid sequence shown in SEQ ID NO: 1, and the second polypeptide, mutant mIFNα4-Fc, had an amino acid sequence shown in the table below.

TABLE 3

| | Amino acid | Coding nucleic acid |
|---|---|---|
| Name | sequence | sequence |
| mIFN-α4 (L30A)-Fc | SEQ ID NO: 37 | SEQ ID NO: 38 |
| mIFN-α4 (R144A)-Fc | SEQ ID NO: 39 | SEQ ID NO: 40 |
| mIFN-α4 (A145G)-Fc | SEQ ID NO: 41 | SEQ ID NO: 42 |
| mIFN-α4 (R149A)-Fc | SEQ ID NO: 43 | SEQ ID NO: 44 |
| mIFN-α4 (S152A)-Fc | SEQ ID NO: 45 | SEQ ID NO: 46 |
| mIFN-α2 (Q124R)-Fc | SEQ ID NO: 47 | SEQ ID NO: 48 |

The ratio of EC₅₀ in PD-L1^{-/-} A20 cells to EC₅₀ in PD-L1^{+/+} A20 cells can reflect the targetability of this bispecific protein. It can be found from the normalization of the ratio with the data of wt-mIFNα4-Fc that R144A and A145G are the two with the optimal targetability, suggesting that these two mutant fusion proteins can more specifically target PD-L1 -positive target cells, while avoiding the induction of IFNAR signaling pathway activation on other cells.

After being fused with the targeting protein, the IFNα mutant with reduced activity can induce IFNAR activation only on target cells. Peripheral off-target is avoided when IFN is used. The mutant IFN-anti-PD-L1 constructed in the present invention has greater potential for treating tumors.

The above descriptions are merely preferred examples of the present invention, and are not intended to limit the present invention. Any modifications, equivalent replacements, improvements, and the like made within the spirit and principle of the present invention shall be all included in the protection scope of the present invention.

What is claimed is:

1. A fusion protein, wherein the fusion protein is an IFN-anti-PD-L1 formed by a fusion of an interferon (IFN) and a PD-L1 binding protein, and the fusion protein is a homodimer protein or a heterodimer protein;
the homodimer protein comprises a first polypeptide and a second polypeptide, the first polypeptide and the second polypeptide of the homodimer protein are identical, and the first polypeptide and the second polypeptide of the homodimer protein comprise the IFN, the PD-L1 binding protein, and an immunoglobulin (Ig) Fc region in sequence from an N-terminus to a C-terminus; and
the heterodimer protein comprises a first polypeptide and a second polypeptide, the first polypeptide and the second polypeptide of the heterodimer protein are different, the first polypeptide of the heterodimer protein comprises the PD-L1 binding protein, and the second polypeptide of the heterodimer protein comprises the Ig Fc region and the IFN located at an N-terminus of the Ig Fc region, an Ig Fc region in the first polypeptide of the heterodimer protein and the Ig Fc region in the second polypeptide of the heterodimer protein are derived from Igs of a same subtype.

2. The fusion protein according to claim 1, wherein the IFN is selected from a type I IFN, a type I IFN mutant, a type II IFN, and/or a type III IFN, comprising IFN-α, IFN-β, IFN-γ, IFN-λ1 (IL-29), IFN-λ2 (IL-28a), IFN-λ (IL-28b), and IFN-ω; the IFN is derived from human or mice.

3. The fusion protein according to claim 1, wherein the Ig Fc region is selected from amino acid sequences of constant regions of IgG1, IgG2, IgG3, and/or IgG4.

4. The fusion protein according to claim 1, wherein the PD-L1 binding protein is selected from an anti-PD-L1 antibody, a single-chain variable fragment (ScFv), an Fab fragment, and an F(ab′)₂ fragment; and the anti-PD-L1 antibody is selected from one or more of Tecentriq, Bavencio, Imfinzi, KN035, CS1001, KL-A167, SHR-1316, YW243.55.S70, ScFv, and YW243.55.S70.

5. The fusion protein according to claim 1, wherein
1) the first polypeptide and the second polypeptide of the homodimer protein comprise the amino acid sequence shown in SEQ ID NO: 3; and
2) the first polypeptide of the heterodimer protein comprises the amino acid sequence shown in SEQ ID NO: 1, and the second polypeptide of the heterodimer protein comprises the amino acid sequence shown in SEQ ID NO: 2, 37, 39, 41, 43, 45, or 47.

6. Isolated nucleic acid molecules encoding the fusion protein according to claim 1, comprising:
1) nucleic acid molecules encoding the homodimer protein having the nucleotide sequences shown in SEQ ID NO: 6; and
2) nucleic acid molecules encoding the heterodimer protein having the nucleotide sequences selected from one or more of SEQ ID NOS: 4, 5, 7, 8, 9, 10, 38, 40, 42, 44, 46, and 48.

7. A vector, comprising the isolated nucleic acid molecules according to claim 6.

8. A method for treating tumors, wherein the method comprises a step of administering to a subject an effective amount of the fusion protein according to claim 1 and/or isolated nucleic acid molecules encoding the fusion protein, wherein the tumor is a tumor against which a PD-1/PD-L1 blockade alone is ineffective; the isolated nucleic acid molecules comprise nucleic acid molecules encoding the homodimer protein having the nucleotide sequences shown in SEQ ID NO: 6 and nucleic acid molecules encoding the heterodimer protein having the nucleotide sequences selected from one or more of SEQ ID NOS: 4, 5, 7, 8, 9, 10, 38, 40, 42, 44, 46, and 48.

9. A pharmaceutical preparation, a pharmaceutical composition, or a kit, wherein the pharmaceutical preparation, the pharmaceutical composition, or the kit comprise the fusion protein according to claim 1.

10. A cell comprising the fusion protein according to claim 1 or nucleic acid molecules encoding the fusion protein, and the cell is used for producing the fusion protein; and the cell is selected from non-human mammalian cells.

11. A method for treating tumors, wherein the method comprises a step of administering to a subject an effective amount of the fusion protein according to claim 1 and an anti-PD-1/PD-L1 antibody together.

12. The method according to claim 11, wherein the tumor is a tumor or an advanced tumor against which a PD-1/PD-L1 blockade alone is ineffective.

13. The method according to claim 11, wherein a patient with the tumor suffers from diseases related to defects or disorders in a transport of peripheral lymphocytes, and the peripheral lymphocytes of the patient do not migrate to tumor tissues.

14. A method for preparing a pharmaceutical composition, a pharmaceutical preparation, or a kit for treating a tumor, comprising using an IFN-α and an anti-PD-L1 antibody together, wherein the IFN-α is intratumourally administered.

15. The method according to claim 14, wherein the tumor is a tumor against which a PD-1/PD-L1 blockade alone is ineffective or a tumor or an advanced tumor against which an IFN alone is ineffective.

16. (canceled)

17. A method for up-regulating an expression of IFN receptors in leukocytes, comprising using the fusion protein according to claim 1, wherein the leukocytes are CD45+ cells, and the IFN receptors are IFNARs.

18. A method for activating DC cells or TIL cells, comprising using the fusion protein according to claim 1.

19. A method for activating tumor-resident T cells, comprising using the fusion protein according to claim 1.

20. An IFN-α4 mutant, wherein the IFN-α4 mutant involves a mutation, wherein the mutation weakens an affinity with a receptor, and the IFN-α4 mutant comprises one or more of mIFN-α4 (L30A) as shown in SEQ ID NO: 25, mIFN-α4 (R144A) as shown in SEQ ID NO: 27, mIFN-α4 (A145G) as shown in SEQ ID NO: 29, mIFN-α4 (R149A) as shown in SEQ ID NO: 31, mIFN-α4 (S152A) as shown in SEQ ID NO: 33, and hIFN-α2 (Q124R) as shown in SEQ ID NO: 35.

21. (canceled)

22. (canceled)


SEQUENCE LISTING (48 sequences)

SEQ ID NO: 1 - protein, 472 aa, synthetic construct
ELDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSARSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 413 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 3 - protein, 660 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAFEEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 4 - DNA, 1482 bp, synthetic construct
  source 1..1482: mol_type = other DNA
atggagacagacacactcctgctatgggtactgctgctctgggttccaggttccactggtgacgagctcgacatccagatgacccagagccccagcagcctgagcgccagcgtgggcgacagagtgaccatcacctgcagagccagccaggacgtgagcaccgccgtggcctggtaccagcagaagcccggcaaggcccccaagctgctgatctacagcgccagcttcctgtacagcggcgtgcccagcagattcagcggcagcggcagcggcaccgacttcaccctgaccatcagcagcctgcagcccgaggacttcgccacctactactgccagcagtacctgtaccaccccgccaccttcggccagggcaccaaggtggagatcaagagaggcggcggcggcagcggcggcggcggcagcggcggcggcggcagcgaggtgcagctggtggagagcggcggcggcctggtgcagcccggcggcagcctgagactgagctgcgccgccagcggcttcaccttcagcgacagctggatccactgggtgagacaggcccccggcaagggcctggagtgggtggcctggatcagcccctacggcggcagcacctactacgccgacagcgtgaagggcagattcaccatcagcgccgacaccagcaagaacaccgcctacctgcagatgaacagcctgagagccgaggacaccgccgtgtactactgcgccagaagacactggcccggcggcttcgactactggggccagggcaccctggtgaccgtgagcgccagatctgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgcccccctgccgcgacgagctgaccaagaaccaggtgagcctgtggtgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 5 - DNA, 1311 bp, synthetic construct
  source 1..1311: mol_type = other DNA
gccgccaccatggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctactagactcattctgcaatgacctccatcagcagctcaatgacctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 6 - DNA, 2052 bp, synthetic construct
  source 1..2052: mol_type = other DNA
atggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctactagactcattctgcaatgacctccatcagcagctcaatgacctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagagtggcggtggtggctccggcggtggtggctccggcggtggtggctccggcggtggtggccgtacggacatccagatgacccagagccccagcagcctgagcgccagcgtgggcgacagagtgaccatcacctgcagagccagccaggacgtgagcaccgccgtggcctggtaccagcagaagcccggcaaggcccccaagctgctgatctacagcgccagcttcctgtacagcggcgtgcccagcagattcagcggcagcggcagcggcaccgacttcaccctgaccatcagcagcctgcagcccgaggacttcgccacctactactgccagcagtacctgtaccaccccgccaccttcggccagggcaccaaggtggagatcaagagaggcggcggcggcagcggcggcggcggcagcggcggcggcggcagcgaggtgcagctggtggagagcggcggcggcctggtgcagcccggcggcagcctgagactgagctgcgccgccagcggcttcaccttcagcgacagctggatccactgggtgagacaggcccccggcaagggcctggagtgggtggcctggatcagcccctacggcggcagcacctactacgccgacagcgtgaagggcagattcaccatcagcgccgacaccagcaagaacaccgcctacctgcagatgaacagcctgagagccgaggacaccgccgtgtactactgcgccagaagacactggcccggcggcttcgactactggggccagggcaccctggtgaccgtgagcgccttcgaagagcccaagagctgcgacaagacccacacctgtcccccttgtcctgcccctgagctgctgggcggacccagcgtgttcctgttccccccaaagcccaaggaccagctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagcccagagaaccccaggtgtacaccctgccccccagcagagatgagctgaccaagaaccaggtgtccctgacctgcctggtcaagggcttctaccccagcgatatcgccgtggagtgggagagcaacggccagcctgagaacaactacaagaccaccccccctgtgctggacagcgatggcagcttcctctacagcaaactgaccgtggacaagagccggtggcagcagggcaacgtgttcagctgcagcgtgttgcacgaggccctgcacaaccactacacccagaagtccctgagcctgagccccggcaaacacgtgtagtaa

SEQ ID NO: 7 - DNA, 1314 bp, synthetic construct
  source 1..1314: mol_type = other DNA
aagcttgccgccaccatggagacagacacactcctgctatgggtactgctgctctgggttccaggttccactggttcgcgaatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaacagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 8 - DNA, 1326 bp, synthetic construct
  source 1..1326: mol_type = other DNA
aagcttgccgccaccatggagacagacacactcctgctatgggtactgctgctctgggttccaggttccactggttcgcgatgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaaagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 9 - DNA, 1329 bp, synthetic construct
  source 1..1329: mol_type = other DNA
aagcttgccgccaccatggagacagacacactcctgctatgggtactgctgctctgggttccaggttccactggttcgcgaatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 10 - DNA, 1224 bp, synthetic construct
  source 1..1224: mol_type = other DNA
aagcttgccgccaccatggagacagacacactcctgctatgggtactgctgctctgggttccaggttccactggttcgcgacacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgcagtggcggtggtggctccggcggtggtggctccggcggtggtggctccggcggtggtggcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 11 - protein, 241 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 12 - DNA, 723 bp, synthetic construct
  source 1..723: mol_type = other DNA
gacatccagatgacccagagccccagcagcctgagcgccagcgtgggcgacagagtgaccatcacctgcagagccagccaggacgtgagcaccgccgtggcctggtaccagcagaagcccggcaaggcccccaagctgctgatctacagcgccagcttcctgtacagcggcgtgcccagcagattcagcggcagcggcagcggcaccgacttcaccctgaccatcagcagcctgcagcccgaggacttcgccacctactactgccagcagtacctgtaccaccccgccaccttcggccagggcaccaaggtggagatcaagagaggcggcggcggcagcggcggcggcggcagcggcggcggcggcagcgaggtgcagctggtggagagcggcggcggcctggtgcagcccggcggcagcctgagactgagctgcgccgccagcggcttcaccttcagcgacagctggatccactgggtgagacaggcccccggcaagggcctggagtgggtggcctggatcagcccctacggcggcagcacctactacgccgacagcgtgaagggcagattcaccatcagcgccgacaccagcaagaacaccgcctacctgcagatgaacagcctgagagccgaggacaccgccgtgtactactgcgccagaagacactggcccggcggcttcgactactggggccagggcaccctggtgaccgtgagcgcc

SEQ ID NO: 13 - protein, 162 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 14 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctactagactcattctgcaatgacctccatcagcagctcaatgacctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggag

SEQ ID NO: 15 - protein, 233 aa, synthetic construct
EPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 16 - DNA, 699 bp, synthetic construct
  source 1..699: mol_type = other DNA
gagcccaagagctgcgacaagacccacacctgtcccccttgtcctgcccctgagctgctgggcggacccagcgtgttcctgttccccccaaagcccaaggaccagctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagcccagagaaccccaggtgtacaccctgccccccagcagagatgagctgaccaagaaccaggtgtccctgacctgcctggtcaagggcttctaccccagcgatatcgccgtggagtgggagagcaacggccagcctgagaacaactacaagaccaccccccctgtgctggacagcgatggcagcttcctctacagcaaactgaccgtggacaagagccggtggcagcagggcaacgtgttcagctgcagcgtgttgcacgaggccctgcacaaccactacacccagaagtccctgagcctgagccccggcaaacacgtg

SEQ ID NO: 17 - protein, 161 aa, synthetic construct
INYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 18 - DNA, 483 bp, synthetic construct
  source 1..483: mol_type = other DNA
atcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaac

SEQ ID NO: 19 - protein, 165 aa, synthetic construct [sequence omitted]

SEQ ID NO: 20 - DNA, 495 bp, synthetic construct
  source 1..495: mol_type = other DNA
tgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaa

SEQ ID NO: 21 - protein, 166 aa, synthetic construct
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 22 - DNA, 498 bp, synthetic construct
  source 1..498: mol_type = other DNA
atgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaac

SEQ ID NO: 23 - protein, 133 aa, synthetic construct
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 24 - DNA, 399 bp, synthetic construct
  source 1..399: mol_type = other DNA
cacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgc

SEQ ID NO: 25 - protein, 162 aa, synthetic construct
CDLPHTYNLGNKRALTVLEEMRRLPPLSCAKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 26 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcgcgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggag

SEQ ID NO: 27 - protein, 162 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIAAEVWRALSSSTNLLARLSEEKE

SEQ ID NO: 28 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcgcagcagaagtctggagagccctctcttcctcaaccaacttgctggcaagactgagtgaggagaaggag

SEQ ID NO: 29 - protein, 162 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRGEVWRALSSSTNLLARLSEEKE

SEQ ID NO: 30 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagaggagaagtctggagagccctctcttcctcaaccaacttgctggcaagactgagtgaggagaaggag

SEQ ID NO: 31 - protein, 162 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWAALSSSTNLLARLSEEKE

SEQ ID NO: 32 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctgggcagccctctcttcctcaaccaacttgctggcaagactgagtgaggagaaggag

SEQ ID NO: 33 - protein, 162 aa, synthetic construct
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALASSTNLLARLSEEKE

SEQ ID NO: 34 - DNA, 486 bp, synthetic construct
  source 1..486: mol_type = other DNA
tgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctcgcatcctcaaccaacttgctggcaagactgagtgaggagaaggag

SEQ ID NO: 35 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKE

SEQ ID NO: 36 - DNA, 495 bp, synthetic construct
  source 1..495: mol_type = other DNA
tgcgatttaccccagacacactctttaggctctcgtaggaccctcatgctgctggcccagatgaggaggatctctttattctcttgtttaaaggacagacacgacttcggcttcccccaagaagagttcggcaaccagttccagaaggctgagaccatccccgtgctgcacgagatgatccagcagatcttcaatttatttagcaccaaggactccagcgccgcttgggatgagactttactggacaagttctacaccgaactgtaccagcagctgaacgatttagaggcttgtgtgatccaaggtgtgggagtgaccgagacccctctgatgaaggaggactccattttagccgtgaggaagtactttcgtaggatcaccctctacctcaaggagaagaagtacagcccttgcgcttgggaggtggtgagggctgagattatgaggagcttctctttatccaccaatctgcaagaatctttaaggtccaaggaa

SEQ ID NO: 37 - protein, 435 aa, synthetic construct
METDTLLLWVLLLWVPGSTGSRCDLPHTYNLGNKRALTVLEEMRRLPPLSCAKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 38 - DNA, 1311 bp, synthetic construct
  source 1..1311: mol_type = other DNA
gccgccaccatggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcgcgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 39 - protein, 435 aa, synthetic construct
METDTLLLWVLLLWVPGSTGSRCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIAAEVWRALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 40 - DNA, 1311 bp, synthetic construct
  source 1..1311: mol_type = other DNA
gccgccaccatggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcgcagcagaagtctggagagccctctcttcctcaaccaacttgctggcaagactgagtgaggagaaggagagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 41 - protein, 435 aa, synthetic construct
METDTLLLWVLLLWVPGSTGSRCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRGEVWRALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 42 - DNA, 1311 bp, synthetic construct
  source 1..1311: mol_type = other DNA
gccgccaccatggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagaggagaagtctggagagccctctcttcctcaaccaacttgctggcaagactgagtgaggagaaggagagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 43 - protein, 436 aa, synthetic construct
METDTLLLWVLLLWVPGSTGSRCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWAALSSSTNLLARLSEEKESSGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 44 - DNA, 1311 bp, synthetic construct
  source 1..1311: mol_type = other DNA
gccgccaccatggagacagacacactcctgctatgggtactgctgctctgggttccaggttccactggttcgcgatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctgggcagccctctcttcctcaaccaacttgctggcaagactgagtgaggagaaggagagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa

SEQ ID NO: 45 - protein, 435 aa, synthetic construct
METDTLLLWVLLLWVPGSTGSRCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWAALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 46 - DNA, 1311 bp, synthetic construct; identical to SEQ ID NO: 44
  source 1..1311: mol_type = other DNA

SEQ ID NO: 47 - protein, 438 aa, synthetic construct
METDTLLLWVLLLWVPGSTGSRCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKHV

SEQ ID NO: 48 - DNA, 1320 bp, synthetic construct
  source 1..1320: mol_type = other DNA
gccgccaccatggagacagacacactcctgctatgggtactgctgctctgggttccaggttccactggttcgcgatgcgatttaccccagacacactctttaggctctcgtaggaccctcatgctgctggcccagatgaggaggatctctttattctcttgtttaaaggacagacacgacttcggcttcccccaagaagagttcggcaaccagttccagaaggctgagaccatccccgtgctgcacgagatgatccagcagatcttcaatttatttagcaccaaggactccagcgccgcttgggatgagactttactggacaagttctacaccgaactgtaccagcagctgaacgatttagaggcttgtgtgatccaaggtgtgggagtgaccgagacccctctgatgaaggaggactccattttagccgtgaggaagtactttcgtaggatcaccctctacctcaaggagaagaagtacagcccttgcgcttgggaggtggtgagggctgagattatgaggagcttctctttatccaccaatctgcaagaatctttaaggtccaaggaaagtggcggtggtggctccggcggtggtggctccggcggtggtggctccggcggtggtggccgtacggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtgcaccctgccccccagccgcgacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagccgctggcagcagggcaacgtgttcagctgcagcgtgatgcacgaggccctgcacaaccactacacccagaagagcctgagcctgagccccggcaagtaa